(19) United States
(12) Patent Application Publication
(10) Pub. No.: US 2006/0051752 A1
(43) Pub. Date: Mar. 9, 2006

(54) One-step multiplex pcr for the identifiation and differentiation of campylobacter species

(76) Inventors: Gehua Wang, Manitoba (CA); Frank G Rodgers, New Hampshire, NH (US)

Correspondence Address: Ade & Company, 1700-360 Main Street, Winnipeg Manitoba, R3C 3Z3, CA

(21) Appl. No.: 10/502,317

(22) PCT Filed: Jan. 7, 2003
(86) PCT No.: PCT/CA03/00041

§ 371 Date: Apr. 22, 2005

Related U.S. Application Data
(60) Provisional application No. 60/350,009, filed on Jan. 23, 2002.
(60) Provisional application No. 60/349,981, filed on Jan. 23, 2002.

(51) Int. Cl. C12Q 1/68; C07H 21/04

(52) U.S. Cl. 435/6; 536/24.1

(57) ABSTRACT

Described herein are a plurality of primers which may be used in a multiplex PCR assay in a fast, accurate, reliable and specific fashion for detecting the presence of specific Campylobacter strains within a sample. These kits can be used on bacterial isolates and has the potential for use directly on foods and environmental samples.

### FIELD OF THE INVENTION

The present invention relates generally to the field of pathogenic organisms. More specifically, the present invention relates to a multiplex PCR-based method for identifying and characterizing Campylobacter species.

### BACKGROUND OF THE INVENTION

The organisms which are referred to as campylobacteria are associated with a diverse range of diseases and habitats and are important from clinical, animal, and economic perspectives. Accurate identification of these organisms is essential for deciding upon appropriate therapeutic measures, and also for furthering our understanding of their pathology and epidemiology.

U.S. Pat. No, 5,981,189 and related U.S. Pat. Nos. 5,695,960 and 6,013,501 disclose the sequence of the hippuricase gene from Campylobacter jejuni and also describe the use of primers or probes derived from within this gene for detection of this Camplyobacter. However, these references do not indicate that any specific primers are preferable nor do they describe the use of multiple primers in a multiplex PCR system for characterizing and identifying multiple Campylobacter strains.

U.S. Pat. No. 6,001,565 teaches a method of identifying Campylobacter strains which involves the PCR amplification of a region conserved between lari, coli, jejuni and upsaliensis followed by cleavage with specific restriction enzymes to produce a diagnostic fragment. As will be apparent to one knowledgeable in the art, this method requires the added step of restriction enzyme digestion.

U.S. Pat. No. 5,691,138 teaches a DNA fragment specific for Campylobacter jejuni. However, use of this fragment alone does not provide any information regarding the presence of other campylobacteria.

U.S. Pat. No. 5,494,795 teaches the use of primers derived from the flagellar genes of Campylobacter coli and Campylobacter jejuni for identification of these strains.

U.S. Pat. No. 5,571,674 teaches primers derived from 16S rRNA for identification of Campylobacter pylori.

U.S. Pat. Nos. 6,166,196 and 6,066,461 teach primers derived from superoxide dismutase gene for detecting Campylobacter jejuni and Campylobacter coli.

Although methods based on DNA probe technology have also been developed, these are not sensitive enough for the detection of Campylobacter spp. in food products. Since Campylobacters have fastidious growth requirements and conventional detection and identification requires at least 4-6 days, the development of fast but reliable detection procedures is needed.

### SUMMARY OF THE INVENTION

According to a first aspect of the invention, there is provided a method for detecting the presence or absence of a Campylobacter strain selected from the group consisting of C. jejuni; C. coli, C. lari; C. upsaliensis; C. fetus and mixtures thereof in a sample comprising:

adding the sample to an amplification mix including at least one primer pair selected from the group consisting of at least 15 contiguous nucleotides of: ACTTCTTTATTGCTTGCTGC (SEQ ID NO. 1) and GCCACAACAAGTAAAGAAGC (SEQ ID NO. 2); GTAAAACCAAAGCTTATCGTG (SEQ ID NO. 3) and TCCAGCAATGTGTGCAATG (SEQ ID NO. 4); TAGAGAGATAGCAAAAGAGA (SEQ ID NO. 5) and TACACATAATAATCCCACCC (SEQ ID NO. 6); AATTGAAACTCTTGCTATCC (SEQ ID NO. 7) and TCATACATTTTACCCGAGCT (SEQ ID NO. 8); GCAAATATAAATGTAAGCGGAGAG (SEQ ID NO. 9) and TGCAGCGGCCCCACCTAT (SEQ ID NO. 10) TATACCGGTAAGGAGTGCTGGAG (SEQ ID NO. 11) and ATCAATTAACCTTCGAGCACCG (SEQ ID NO. 12);

incubating the amplification mixture under conditions promoting nucleic acid amplification; and

detecting the amplification product.

According to a second aspect of the invention there is provided pair of amplification primers selected from the group consisting of at least 15 contiguous nucleotides: ACTTCTTTATTGCTTGCTGC (SEQ ID NO. 1) and GCCACAACAAGTAAAGAAGC (SEQ ID NO. 2); GTAAAACCAAAGCTTATCGTG (SEQ ID NO. 3) and TCCAGCAATGTGTGCAATG (SEQ ID NO. 4); TAGAGAGATAGCAAAAGAGA (SEQ ID NO. 5) and TACACATAATAATCCCACCC (SEQ ID NO. 6); AATTGAAACTCTTGCTATCC (SEQ ID NO. 7) and TCATACATTTTACCCGAGCT (SEQ ID NO. 8); GCAAATATAAATGTAAGCGGAGAG (SEQ ID NO. 9) and TGCAGCGGCCCCACCTAT (SEQ ID NO. 10) TATACCGGTAAGGAGTGCTGGAG (SEQ ID NO. 11) and ATCAATTAACCTTCGAGCACCG (SEQ ID NO. 12); and mixtures thereof.

According to a third aspect of the invention, there is provided a kit comprising at least one primer pair selected from the group consisting of at least 15 contiguous nucleotides of: ACTTCTTTATTGCTTGCTGC (SEQ ID NO. 1) and GCCACMCAAGTAAAGAAGC (SEQ ID NO. 2); GTAAAACCAAAGCTTATCGTG (SEQ ID NO. 3) and TCCAGCAATGTGTGCAATG (SEQ ID NO. 4); TAGAGAGATAGCAAAAGAGA (SEQ ID NO. 5) and TACACATAATAATCCCACCC (SEQ ID NO. 6); AATTGAAACTCTTGCTATCC (SEQ ID NO. 7) and TCATACATTTTACCCGAGCT (SEQ ID NO. 8); GCAAATATAAATGTAAGCGGAGAG (SEQ ID NO. 9) and TGCAGCGGCCCCACCTAT (SEQ ID NO. 10) TATACCGGTAAGGAGTGCTGGAG (SEQ ID NO. 11) and ATCAATTAACCTTCGAGCACCG (SEQ ID NO. 12) and mixtures thereof.

### BRIEF DESCRIPTION OF THE DRAWINGS

FIG. 1 shows a amplification products prepared from samples using the multiplex PCR system.

TABLE 1 shows the primer sequences and expected sizes of the products.

TABLE 2 shows the predicted sizes of restriction fragments and enzymes used for RFLP analysis of amplified products.

TABLE 3 shows PCR results by multiplex PCR analysis of Campylobacter strains.

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

Definitions

As used herein, “amplification reaction mixture” or “amplification mixture” refers to an aqueous solution comprising the various reagents used to amplify a target nucleic acid. These include but are by no means limited to enzymes, aqueous buffers, salts, target nucleic acid and nucleoside triphosphates.

As used herein, “isolated” or “substantially pure”, when referring to nucleic acids, refers to those which have been purified away from other cellular components and/or contaminants by standard techniques, for example, column chromatography, CsCl banding, and alkaline/SDS treatment as well as other techniques well known in the art.

As used herein, “DNA sequence” refers to a single-stranded or double-stranded DNA polymer composed of the nucleotide bases, adenosine, thymidine, cytosine and guanosine.

As used herein, “nucleotide polymerase” refers to enzymes that are capable of catalyzing the synthesis of DNA or RNA from nucleoside triphosphate precursors.

As used herein, “primer” refers to an oligonucleotide capable of acting as a point of initiation of DNA synthesis under conditions in which synthesis of a primer extension product complementary to a nucleic acid strand is initiated.

Proper annealing conditions depend, for example, on the length of the primer or probe, the base composition of said primer or probe and the number of mismatches present and their relative position.

As discussed above, Campylobacter spp. are difficult to detect using conventional methods. Therefore a PCR procedure based on the amplification of specific genes was developed that specifically identifies the four major Campylobacter species, as described below. This assay provides an excellent tool for the rapid and sensitive identification of isolates of Campylobacter spp. and may also serve to identify these pathogens from chicken samples or clinical isolates directly.

Described herein are a plurality of primers which may be used in a multiplex PCR assay in a fast, accurate, reliable and specific fashion for detecting the presence of specific Campylobacter strains within a sample. These kits can be used on bacterial isolates and has the potential for use directly on foods and environmental samples.

Surveillance for pathogens and early identification of outbreaks are also critical for reducing the incidence of foodborne disease. The Canadian National Laboratory for Enteric Pathogens (NLEP) uses surveillance and laboratory-based epidemiologic markers for specific bacteria strains to track human infections and to identify and characterize outbreaks.

Newer molecular models are urgently required for use in clinical and laboratory medicine as well as in the environmental, abbatoir and food industry arenas, to assist in resolving the problem of Campylobacter disease.

As will be known to one of skill in the art, DNA amplification involves allowing two primers to anneal to opposite strands of a template DNA in an amplification mixture and allowing extension of the primers. This process is repeated several times, thereby producing an amplification product. The PCR process is discussed in detail in for example U.S. Pat. No. 4,199,559, U.S. Pat. No. 4,683,195 and U.S. Pat. No. 4,683,202, which are incorporated herein by reference.

To begin the PCR process, the target nucleic acid in the sample is denatured, typically by heating. Once the strands are separated, the next step involves hybridizing the separated strands with the amplification primers. The primers are then extended to form complementary copies of the target strands, and the cycle of denaturation , hybridization and extension is repeated as many times as necessary to obtain the desired amount of amplified nucleic acid.

Template-dependent extension of primers in PCR is catalyzed by a polymerizing agent in the presence of adequate amounts of four deoxyribonucleotide triphosphates in a reaction medium. Suitable polymerizing agents are enzymes known to catalyze template-dependent DNA synthesis. For example, if the template is RNA, a suitable polymerizing agent to convert RNA to cDNA is reverse transcriptase, such as avian myeloblastosis virus RT or Murine Moloney Leukemia Virus RT. If the template is DNA, suitable polymerases include for example E. coli DNA polymerase I, the Klenow fragment of DNA polymerase I, T₄ DNA polymerase, Hot Tub® and Taq polymerase.

A preferred mode for carrying out the PCR reaction is the multiplex mode. The multiplex mode involves the. simultaneous amplification of different target regions using more than one set of PCR primers. As will be apparent to one of skill in the art, increasing the number of distinct primers in an amplification mixture can result in production of non-diagnostic bands. As such, the nucleic acid composition, location of the primers within the genome and length of the primers selected is critical for functioning of this method.

Referring to Table 1, the primers for use in the multiplex PCR system described herein comprise at least 15 contiguous nucleotides of the following:

ACTTCTTTATTGCTTGCTGC, designated hereafter as SEQ ID NO. 1 or CJF. As can be seen, these sequence corresponds to nucleotides 1662-1681 of Genbank accession No. Z36940.

GCCACAACAAGTAAAGAAGC, designated hereafter as SEQ ID NO. 2 or CJR. As can be seen, these sequence corresponds to nucleotides 1984-1965 of Genbank accession No. Z36940.

GTAAAACCAAAGCTTATCGTG, designated hereafter as SEQ ID NO. 3 or CCF. As can be seen, these sequence corresponds to nucleotides 337-357 of Genbank accession No. AF136494.

TCCAGCAATGTGTGCAATG, designated hereafter as SEQ ID NO. 4 or CCR. As can be seen, these sequence corresponds to nucleotides 462-444 of Genbank accession No. AF136494.

TAGAGAGATAGCAAAAGAGA, designated hereafter as SEQ ID NO. 5 or CLF. As can be seen, these sequence corresponds to nucleotides 318-337 of Genbank accession No. AF136495.

TACACATAATAATCCCACCC, designated hereafter as SEQ ID NO. 6 or CLR. As can be seen, these sequence corresponds to nucleotides 568-549 of Genbank accession No. AF136495.

AATTGAAACTCTTGCTATCC, designated hereafter as SEQ ID NO. 7 or CUF. As can be seen, these sequence corresponds to nucleotides 63-82 of Genbank accession No. AF136496.

TCATACATTTTACCCGAGCT, designated hereafter as SEQ ID NO. 8 or CUR. As can be seen, these sequence corresponds to nucleotides 266-247 of Genbank accession No. AF1 36496.

GCAAATATAAATGTAAGCGGAGAG, designated hereafter as SEQ ID NO. 9 or CFF. As can be seen, these sequence corresponds to nucleotides 2509-2532 of Genbank accession No. AF048699.

TGCAGCGGCCCCACCTAT, designated hereafter as SEQ ID NO. 10 or CFR. As can be seen, these sequence corresponds to nucleotides 2943-2926 of Genbank accession No. AF048699.

TATACCGGTAAGGAGTGCTGGAG, designated hereafter as SEQ ID NO. 11 or 23SF. As can be seen, these sequence corresponds to nucleotides 3807-3829 of Genbank accession No. Z29326.

ATCAATTAACCTTCGAGCACCG, designated hereafter as SEQ ID NO. 12 or 23SR. As can be seen, these sequence corresponds to nucleotides 4456-4435 of Genbank accession No. Z29326.

As will be appreciated by one of skill in the art, the primers may comprise at least 16 contiguous nucleotides, that is, 16 or more contiguous nucleotides, at least 17 contiguous nucleotides or at least 18 contiguous nucleotides of any one of the above-described primers. In yet other embodiments, the primers may consist essentially of at least 15 contiguous nucleotides, at least 16 contiguous nucleotides, at least 17 contiguous nucleotides or at least 18 contiguous nucleotides of any one of the above-described primers. As will be appreciated by one of skill in the art, in this context, “consists essentially of” indicates that the primer consists of those nucleotides only but may also include other components which do not materially affect the functioning of the primer (that is, its ability to hybridize to its target sequence). These include for example but are by no means limited to labels, universal bases, tags and the like known in the art.

As described below, the last two primers (SEQ ID Nos 11 and 12) are used as positive controls in some embodiments. As will be apparent to one of skill in the art, other suitable primers which generate an amplification product without producing background or false positive products may also be used as positive controls and are within the scope of the invention.

In use, a sample suspected of Campylobacter contamination is prepared for PCR analysis. As will be appreciated by one knowledgeable in the art, samples may be selected from any source wherein Campylobacter contamination is suspected, for example, but by no means limited to, fecal samples, environmental samples, veterinary samples, medical diagnostic samples and food samples. In some embodiments, the sample may be incubated under conditions known in the art which promote amplification of bacteria prior to preparation for PCR analysis.

The sample is then mixed with at least one of the primer pairs described above as well as amplification enzymes, aqueous buffers, salts, target nucleic acid and nucleoside triphosphates as discussed above, thereby forming an amplification mixture. The amplification mixture is then subjected to conditions suitable for nucleic acid amplification. Specifically, as discussed above, nucleic acid in the sample is denatured by heating. Once the strands are separated, the temperature of the sample is lowered and the amplification primers hybridize to their target DNA. The temperature is elevated and the primers are then extended to form complementary copies of the target strands. The cycle of denaturation, hybridization and extension is repeated as many times as necessary to obtain the desired amount of amplified nucleic acid.

The amplification products generated as described above may be detected by any suitable means known in the art, for example, by a characteristic size as detected on a polyacrylamide or agarose gel stained with ethidium bromide. Alternatively, amplified products may be detected by a labeled probe. The label may be for example a radiolabel or a fluorescent or chemiluminescent label. Examples of detection methods known in the art include but are by no means limited to U.S. Pat. No. 6,245,514 and U.S. Pat. No. 6,117,635, both of which are incorporated herein by reference.

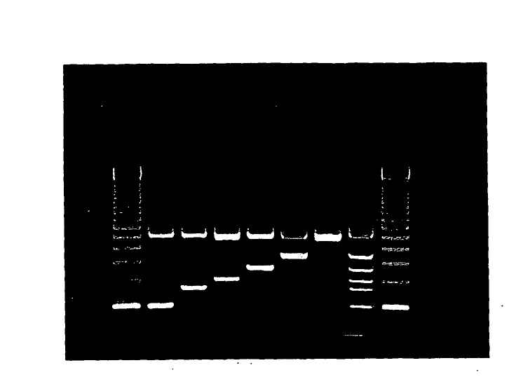

As discussed above, the amplification mixture may contain at least one of the above-described primer pairs. Specifically, the presence of an approximately 323 bp amplification product when primer pair CJF/CJR is used indicates that the sample contains Campylobacter jejuni; the presence of an approximately 126 bp amplification product when using CCF/CCR indicates that the sample contains Campylobacter coli; the presence of an approximately 251 bp amplification product when primer pair CLF/CLR is used indicates that the sample contains Campylobacter lari; the presence of an approximately 204 bp band when primer pair CUF/CUR is used indicates that the sample contains Campylobacter upsaliensis; and the presence of a 435 bp amplification product when primer pair CFF/CFR indicates that the sample contains Campylobacter fetus, as shown in FIG. 1. In some embodiments, primer pair 23SF/23SR may be included as a positive control. As will be appreciated by one knowledgeable in the art, other suitable positive controls may also be used.

As will be appreciated by one knowledgeable in the art, the exact sizes of the amplification products described above may vary somewhat depending on the specific sequences of the primer pairs utilized.

For commercial convenience, any one or any combination of the above-described primers may be packaged in the form of a kit. That is, the kit will include at least one primer and instructions for use of the kit, for example, reaction conditions, sample preparation and the like. Reagents for performing a nucleic acid amplification reaction may also be included with the amplification primers, for example, buffers, additional primers, positive and negative controls, nucleoside triphosphates, enzymes, and instructions. Specifically, the kit may include primers or primer pairs selected from the group consisting of: CJF/CJR; CCF/CCR; CLF/CLR; CUF/CUR; CFF/CFR; and combinations thereof.

As discussed below, in one embodiment, the PCR products are visualized on a gel following electrophoretic separation. As will be appreciated by one knowledgeable in the art, in some embodiments, detection of the bands may be automated wherein the samples are loaded onto a suitable separating system and bands are detected automatically. Examples of such techniques may be found in for example U.S. Pat. No. 5,840,877, U.S. Pat. No. 4,930,893, U.S. Pat. No. 6,005,663, U.S. Pat. No. 5,710,628, U.S. Pat. No. 5,543,018 and U.S. Pat. No. 5,190,632, which are incorporated herein by reference.

It is of note that any amplification protocol which utilizes cyclic, specific hybridization of primers to the target sequence, extension of the primers using the target sequence as a template and separation or displacement of the extension products from the target sequence may employ the amplification primers described herein.

The invention will now be described by way of examples. However, the invention is not limited to the examples.

### EXAMPLE I

### Bacterial Strains And Culture Media

A total of 131 strains of various species of enterobacteria were used in this study. Of these, 127 strains were campylobacters: 70 C. jejuni subsp jejuni; 21 C. coli; 7 C. lari; 6 each of C. upsaliensis, C. fetus subsp fetus, C. fetus subsp venerealis and C. fetus subsp hyointestinalis; one each of C. s. bubulus and C. fecalis; 3 Helicobacter pylori, 2 Escherichia coli and 2 Aeromonas hydrophilia strains. All strains were obtained from the culture collection of the National Laboratory Enteric Pathogen (NLEP). The Campylobacter isolates were grown on Mueller-Hinton agar (Oxoid, Hampshire, England) supplemented with 10% sheep blood. Inoculated plates were incubated at 37° C. in a microaerobic atmosphere containing 5% O₂, 10% CO₂ and 85% N₂.

### EXAMPLE II

### DNA Template Preparation

Total DNA was prepared by whole cell procedure. Templates for PCR were prepared using half loopfulls of culture that were transferred to 1 ml Brain Hart Infusion (BHI) broth. The optical density was adjusted to give 0.5 at A600. The whole cell DNA preparations were diluted 1:500 in distilled water and were then heated at 100° C. for 10 min in a 0.5 ml Eppendorf™ tube. The templates were used immediately for PCR reactions or were kept at 4° C. for up to 1 month.

### EXAMPLE III

### Multiplex PCR Conditions

The multiplex PCR reaction tube contained 200 μM deoxynucleoside triphosphate; 2.5 μl of 10X reaction buffer (500 mM Tris-HCl[pH 8.3], 100 mM KCl, 50 mM [NH₄]₂SO₄); 2.0 mM MgCl₂, 0.5 μM CJF/CJR, CCF/CCR, and CLF/CLR primers; 1 μM CUF/CUR; and CFF/CFR primers and 0.2 μM 23S rRNA primers; 1.25 U of FastStart Taq™ DNA Polymerase (Roche Diagnostic, GmbH, Germany) and 2.5 μl whole cell template DNA. The volume of this mix was adjusted to 25 μl with sterile distilled water. DNA amplification was carried out in a Perkin-Elmer thermocycler under the following conditions: an initial denaturation step at 95° C. for 6 min, followed by 30 cycles of amplification (denaturation at 95° C. for 0.5 min, annealing at 59° C. for 0.5 min and extenstion at 72° C. for 0.5 min), ending with a final extension at 72° C. for 7 minutes. It is of note that other suitable temperatures and times may also be used.

### EXAMPLE IV

### Results

FIG. 1 shows the presence of the amplified products after 1.5% agarose gel electrophoresis, when the representative Campylobacter reference strains were used as the templates for the PCR reaction. Following the multiplex primer and amplification steps, 6 bands were obtained from a mixture of DNA containing each of the 5 campylobacter spp. (lane 8, FIG. 1). The amplicons from the control strains were subjected to further confirmation and characterization by digestion with restriction endonucleases with cleavage sites within the amplicon. The restriction enzymes used and the predicted product sizes are shown in Table 2.

Among 131 tested samples, all the non-reference Campylobacter species or subspecies were identified by biotyping and 16S rRNA-PCR(Marshall et al., 1999, J. Clin. Microbiol. 37: 4158-4160). Full agreement for the species-specific primers was observed for C. jejuni, C. coli, C. lari, C. upsaliensis and C. fetus subsp fetus. The primer for Campylobacter 23S rRNA was presented in all tested Campylobacter strains but failed to amplify E. coli and A. hydrophila strains, which further validated the specificity of the assay (Table 3). The sensitivity of the colony PCR was 1-10,000 cfu for C. jejuni, 50-5,000 cfu for C. coli, 5-5,000 for C. lari, 5-3,000 for C. upsaliensis and 10-1,000 for C. fetus subsp fetus.

### EXAMPLE V

### Discussion

Human campylobacteriosis is largely a foodborne infection in which foods of animal origin, particularly poultry, play an important role. Case control studies have suggested that a major source of human infection is the handling and consumption of contaminated poultry meat (Tauxe, 1992, in Campylobacter jejuni. Current status and Future Trends (American Society for Microbiology: Washington), pp 9-19). One study indicated that Campylobacter species were isolated from 73.2% of 489 meat samples studies (Kramer et al., 2000, J. Food Prot 63: 1654-1659).

Clinically the most important campylobacters are the members of the thermophilic group, including C. jejuni, C. coli, C. lari, and C. upsaliensis, with C. jejuni responsible for the majority of human cases (Allos and Blaser, 1995, Clin Infect Dis 20: 1092-1099). Campylobacter fetus, also recognized as a human and animal pathogen, has been identified in 12.5% of ox liver samples (Kramer et al., 2000). Accurate identification of these organisms is needed in order to decide upon appropriate therapeutic measures, to understand the pathology of disease and to provide clinical and epidemiological data for disease control.

As discussed above, there are a number of protocols described in the literature (Blom et al., 1995, J Clin Microbiol 33: 1360-1362; Casademont et al., 1998, FEMS Immunol Med Microbiol 21: 269-281; Casademont et al., 2000, Mol Cell Probes 14: 233-240; Chuma et al., 2000, J Vet Med Sci 62: 1291-1295; Denis et al., 1999, Lett Appl Microbiol 29: 406-410; Fermer and Engvall, 1999, J Clin Microbiol 37: 3370-3373; Gonzalez et al., 1997, J Clin Microbiol 35: 759-763; Oyarzabal et al., 1997, Vet Microbiol 58: 61-71; Steinhauserova et al., 2001, Appl Microbiol 90: 470-475; and van Doorn et al., 1999, J Clin Microbiol 37: 1790-1796) to differentiate the closely related thermophilic C. jejuni, C. coli, C. lari and C. upsaliensis species as well as C. fetus. Most of these methods are based on DNA probe technology or rRNA PCR-RFLP, neither of which are simple enough for the detection of Camplyobacter species in food products and environmental samples. This is due to the requirement of restriction enzymes or hybridization steps with species-specific probes that follow PCR amplification. A combination of PCR and hybridization was developed for the rapid detection of C. fetus but was not capable of differentiating C. fetus subsp fetus from C. fetus subsp venerealis (Casademont et al., 2000). A recent study describes the use of multiplex PCR assays for simultaneously differentiating C. jejuni, C. coi, and C. lari (Chuma et al., 2000). These were based on the sequence of a gene possibly encoding an oxidoreductase subunit of C. jejuni (Winters et al., 1997, Mol Cell Probes 11: 267-271, Genbank accession No. AL139075), aspartokinase gene for C. coli (Linton et al., 1997, J. Clin Microbiol 35: 2568-2572) and 16S rRNA for C. lari (Oyarzabal et al., 1997, J Microbio Methods 29: 97-102).

PCR and PCR-RFLP study indicated that hipO encoding C. jejuni hippuricase gene was unique and highly conserved to C. jejuni. Detection of hipO by PCR provided a useful identification marker for C. jejuni (Slater and Owen, 1997, Lett Appl Microbiol 25: 274-278; Steinhauserova et al., 2001, Appl Microbiol 90: 470475). In addition, PCR-hybridization confirmed that Campylobacter glyA gene can be used as the target to identify and differentiate C. jejuni, C. coli, C. lari, and C. upsaliensis at the species level (Al Rashid et al, 2000, J Clin Microbiol 38: 1488-1494). In addition, the C. fetus subsp fetus sapB2 gene was recognized as a suitable target to identify C. fetus (Casademont et al, 1998).

In this study, we have developed a colony multiplex PCR-based diagnostic protocol to simultaneously detect 5 Campylobacter genes: hipO, C. coli-glyA, C. lari-glyA, C. upsaliensis-glyA and sapB2 for C. fetus subsp fetus. As an internal control, Campylobacter 23S rRNA was found in all of the Campylobacter subsp and H. pylori strains studied, which provided additional validation for monitoring the multiplex PCR conditions and PCR reagents including DNA template quality.

As can be seen, this method can be used for the detection and identification of thermophilic campylobacters in complex samples, such as foods in which low numbers are present. This method can also complement or replace phenotypic methods for identifying thermophilic Campylobacter species. Thus, as discussed above, in some embodiments, the invention is a diagnostic kit for Campylobacter species-level identification. The multiplex primers are specific for C. jejuni, C. coli, C. lari, C. upsaliensis and C. fetus subsp fetus since no amplification product was obtained when either E. coli or A. hydrophila DNA was used as the template. Thus, the above-described PCR assay offers an alternative to traditional biochemical typing methods for the identification and differentiation of C. jejuni, C. coli, C. lari, C. upsaliensis and C. fetus subsp fetus. The method is accurate, simple to perform and can be completed within 3 hours.

While the preferred embodiments of the invention have been described above, it will be recognized and understood that various modifications may be made therein, and the appended claims are intended to cover all such modifications which may fall within the spirit and scope of the invention.

TABLE 1

Primer sequences used in the multiplex PCR assay and the expected sizes
of the products

| | Size | | Genbank | | gene |
|---|---|---|---|---|---|
| Primer | (bp) | Sequence (5′-3′) | Accession No | Target gene | location |
| CJF | 323 | ACTTCTTTATTGCTTGCTGC | Z36940 | C. jejuni hipO | 1662-1681 |
| CJR | | GCCACAACAAGTAAAGAAGC | | | 1984-1965 |
| CCF | 126 | GTAAAACCAAAGCTTATCGTG | AF136494 | C. coli glyA | 337-357 |
| CCR | | TCCAGCAATGTGTGCAATG | | | 462-444 |
| CLF | 251 | TAGAGAGATAGCAAAAGAGA | AF136495 | C. lari glyA | 318-337 |
| CLR | | TACACATAATAATCCCACCC | | | 568-549 |
| CUF | 204 | AATTGAAACTCTTGCTATCC | AF136496 | C. upsaliensis glyA | 63-82 |
| CUR | | TCATACATTTTACCCGAGCT | | | 266-247 |
| CFF | 435 | GCAAATATAAATGTAAGCGGAGAG | AF048699 | sapB2 | 2509-2532 |
| CFR | | TGCAGCGGCCCCACCTAT | | | 2943-2926 |
| 23SF | 650 | TATACCGGTAAGGAGTGCTGGAG | Z29326 | C. jejuni 23S rRNA | 3807-3829 |
| 23SR | | ATCAATTAACCTTCGAGCACCG | | | 4456-4435 |

TABLE 2
Predicted sizes of restriction fragments and enzymes
used for restriction fragment length polymorphism
analysis of amplified products of multiplex PCR

| | | | Expected size |
|---|---|---|---|
| | PCR amplicon | | of restriction |
| genes | sizes(bp) | Enzymes | fragments(bp) |
| hipO | 323 | BsrDI | 109, 214 |
| C. coli glyA | 126 | AluI | 11, 36, 79 |
| C. lari glyA | 251 | ApoI | 79, 172 |
| C. upsaliensis glyA | 204 | DdeI | 31, 173 |
| C. fetus fetus-sapB2 | 435 | BclI | 130, 305 |
| C. jejuni 23S rRNA | 650 | HhaI | 212, 438 |

TABLE 3
PCR results by multiplex PCR analysis of Campylobacter strains

| | | PCR positive for | | | | | |
|---|---|---|---|---|---|---|---|
| | | | | | C. | C. | |
| | Tested | C. | C. | C. | upsali- | fetus | |
| Strains | No. | jejuni | coli | lari | ensis | fetus | 23S |
| C. jejuni | 70 | 70 | —* | — | — | — | 70 |
| C. coli | 21 | — | 21 | — | — | — | 21 |
| C. lari | 7 | — | — | 7 | — | — | 7 |
| C. upsaliensis | 6 | — | — | — | 6 | — | 6 |
| C. fetus fetus | 6 | — | — | — | — | 6 | 6 |
| C. fetus | 6 | — | — | — | — | — | 6 |
| hyointestinalis | | | | | | | |
| C. fetus | 6 | — | — | — | — | — | 6 |
| venerealis | | | | | | | |
| C. s. bubulus | 1 | — | — | — | — | — | 1 |
| C. s. fecalis | 1 | — | — | — | — | — | 1 |
| H. pylori | 3 | — | — | — | — | — | 3 |
| E. coli | 2 | — | — | — | — | — | — |
| A. hydrophila | 2 | — | — | — | — | — | — |
| Total | 131 | 70 | 21 | 7 | 6 | 6 | 127 |

*Negative

1. A method for detecting the presence or absence of a Campylobacter strain selected from the group consisting of C. jejuni; C. coli; C. lari; C. upsaliensis; C. fetus spp fetus and mixtures thereof in a sample comprising:
adding the sample to an amplification mix including at least one primer pair selected from the group consisting of at least 15 contiguous nucleotides of: ACTTCTTTATTGCTTGCTGC (SEQ ID NO. 1) and GCCACAACAAGTAAAGAAGC (SEQ ID NO. 2); GTAAAACCAAAGCTTATCGTG (SEQ ID NO. 3) and TCCAGCAATGTGTGCAATG (SEQ ID NO. 4); TAGAGAGATAGCAAAAGAGA (SEQ ID NO. 5) and TACACATAATAATCCCACCC (SEQ ID NO. 6); AATTGAAACTCTTGCTATCC (SEQ ID NO. 7) and TCATACATTTTACCCGAGCT (SEQ ID NO. 8); GCAAATATAAATGTAAGCGGAGAG (SEQ ID NO. 9) and TGCAGCGGCCCCACCTAT (SEQ ID NO. 10) TATACCGGTAAGGAGTGCTGGAG (SEQ ID NO. 11) and ATCAATTAACCTTCGAGCACCG (SEQ ID NO. 12);
incubating the amplification mixture under conditions promoting nucleic acid amplification; and
detecting the amplification product:

2. A pair of amplification primers selected from the group consisting of at least 15 contiguous nucleotides of: ACTTCTTTATTGCTTGCTGC (SEQ ID NO. 1) and GCCACAACAAGTAAAGAAGC (SEQ ID NO. 2); GTAAAACCAAAGCTTATCGTG (SEQ ID NO. 3) and TCCAGCAATGTGTGCAATG (SEQ ID NO. 4); TAGAGAGATAGCAAAAGAGA (SEQ ID NO. 5) and TACACATAATAATCCCACCC (SEQ ID NO. 6); AATTGAAACTCTTGCTATCC (SEQ ID NO. 7) and TCATACATTTTACCCGAGCT (SEQ ID NO. 8); GCAAATATAAATGTAAGCGGAGAG (SEQ ID NO. 9) and TGCAGCGGCCCCACCTAT (SEQ ID NO. 10); TATACCGGTAAGGAGTGCTGGAG (SEQ ID NO. 11) and ATCAATTAACCTTCGAGCACCG (SEQ ID NO. 12); and mixtures thereof.

3. A kit comprising at least one primer selected from the group consisting of at least 15 contiguous nucleotides of:
ACTTCTTTATTGCTTGCTGC (SEQ ID NO. 1) and GCCACAACAAGTAAAGAAGC (SEQ ID NO. 2); GTAAAACCAAAGCTTATCGTG (SEQ ID NO. 3) and TCCAGCAATGTGTGCAATG (SEQ ID NO. 4); TAGAGAGATAGCAAAAGAGA (SEQ ID NO. 5) and TACACATAATAATCCCACCC (SEQ ID NO. 6); AATTGAAACTCTTGCTATCC (SEQ ID NO. 7) and TCATACATTTTACCCGAGCT (SEQ ID NO. 8); GCAAATATAAATGTAAGCGGAGAG (SEQ ID NO. 9) and TGCAGCGGCCCCACCTAT (SEQ ID NO. 10); TATACCGGTAAGGAGTGCTGGAG (SEQ ID NO. 11) and ATCAATTAACCTTCGAGCACCG (SEQ ID NO. 12) and mixtures thereof.


SEQUENCE LISTING (12 sequences)

SEQ ID NO: 1 - DNA, 20 bp, campylobacter
acttctttattgcttgctgc

SEQ ID NO: 2 - DNA, 20 bp, campylobacter
gccacaacaagtaaagaagc

SEQ ID NO: 3 - DNA, 21 bp, campylobacter
gtaaaaccaaagcttatcgtg

SEQ ID NO: 4 - DNA, 19 bp, campylobacter
tccagcaatgtgtgcaatg

SEQ ID NO: 5 - DNA, 20 bp, campylobacter
tagagagatagcaaaagaga

SEQ ID NO: 6 - DNA, 20 bp, campylobacter
tacacataataatcccaccc

SEQ ID NO: 7 - DNA, 20 bp, campylobacter
aattgaaactcttgctatcc

SEQ ID NO: 8 - DNA, 20 bp, campylobacter
tcatacattttacccgagct

SEQ ID NO: 9 - DNA, 24 bp, campylobacter
gcaaatataaatgtaagcggagag

SEQ ID NO: 10 - DNA, 18 bp, campylobacter
tgcagcggccccacctat

SEQ ID NO: 11 - DNA, 23 bp, campylobacter
tataccggtaaggagtgctggag

SEQ ID NO: 12 - DNA, 22 bp, campylobacter
atcaattaaccttcgagcaccg